(19) United States
(12) Patent Application Publication
(10) Pub. No.: US 2009/0286797 A1
(43) Pub. Date: Nov. 19, 2009

(54) Novel Quinoxaline Derivatives and Their Medical Use

(76) Inventors: Dan Peters, Ballerup (DK); Jeppe Kejser Christensen, Ballerup (DK); Kasper Harpsøe, Kobenhavn (DK); Tommy Liljefors, København (DK)

Correspondence Address: BIRCH STEWART KOLASCH & BIRCH, PO BOX 747, FALLS CHURCH, VA, 22040-0747, US

(21) Appl. No.: 12/085,144

(22) PCT Filed: Nov. 20, 2006
(86) PCT No.: PCT/EP2006/068643

§ 371 Date: May 16, 2008

Related U.S. Application Data
(60) Provisional application No. 60/741,880, filed on Dec. 5, 2005.

(30) Foreign Application Priority Data
Nov. 22, 2005 (DK) PA 2005 01639
Mar. 15, 2006 (DK) PA 2006 00369

(51) Int. Cl. A61K 31/55; C07D 401/06; A61K 31/498

(52) U.S. Cl. 514/249; 544/353; 514/217.07; 540/597

(57) ABSTRACT

This invention relates to novel quinoxaline derivatives having medical utility, to use of the quinoxaline derivatives of the invention for the manufacture of a medicament, to pharmaceutical compositions comprising the quinoxaline derivatives of the invention, and to methods of treating a disorder, disease or a condition of a subject, which disorder, disease or condition is responsive to positive modulation of AMPA receptor mediated synaptic responses.

### TECHNICAL FIELD

This invention relates to novel quinoxaline derivatives having medical utility, to use of the quinoxaline derivatives of the invention for the manufacture of a medicament, to pharmaceutical compositions comprising the quinoxaline derivatives of the invention, and to methods of treating a disorder, disease or a condition of a subject, which disorder, disease or condition is responsive to positive modulation of AMPA receptor mediated synaptic responses.

### BACKGROUND ART

L-Glutamate is the major excitatory neurotransmitter in the mammalian central nervous system, which activates several subtypes of ionotropic and metabotropic receptors. The ionotropic receptors can be divided into three subtypes, which are defined by the depolarizing actions of the selective agonists N-methyl-D-aspartate (NMDA), alpha-amino-3-hydroxy-5-methylisoxazole-4-propionic acid (AMPA), and kainic acid (KA).

AMPA receptors are assembled from four protein sub-units known as GluR1 to GluR4, while kainic acid receptors are assembled from the sub-units GluR5 to GluR7, and KA-1 and KA-2.

AMPA receptors have been associated with diseases and conditions as diverse as cognitive deficits, psychotic disorders, sexual dysfunction, schizophrenia, depression, autism, Alzheimer's disease, attention deficit and senile dementia, or from a disorder or disease resulting from trauma, from stroke, from epilepsy, from Alzheimer's disease, from a neurotoxic agent, from aging, from a neurodegenerative disorder, from alcohol intoxication, from substance abuse, from cardiac bypass surgery or from cerebral ischemia.

WO 94/02475 describes aniracetam analogues including certain quinoxaline derivatives useful as modulators of AMPA receptor mediated synaptic responses. However the quinoxaline derivatives of the present invention are not disclosed.

### SUMMARY OF THE INVENTION

It is an object of the present invention to provide new quinoxaline derivatives useful as modulators of the AMPA sensitive glutamate receptors. More particularly it is an object to provide new quinoxaline derivatives showing a less profound desensitization of glutamate receptors.

In its first aspect the invention provides novel quinoxaline derivatives characterised by Formula I

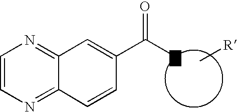

any of its enantiomers or any mixture of its enantiomers, or a pharmaceutically-acceptable addition salt thereof, or an N-oxide thereof, wherein the circular line designates an N-containing heterocyclic 5-8 membered ring; and R′ represents 2- or 3-alkyl, cycloalkyl, hydroxy, alkoxy, alkoxy-alkyl, hydroxy-alkyl or carbamoyl.

In another aspect the invention provides a pharmaceutical composition comprising a therapeutically effective amount of the quinoxaline derivative of the invention.

Viewed from another aspect the invention relates to the use of the quinoxaline derivative of the invention for the manufacture of a pharmaceutical composition.

In a final aspect the invention provides a method of treatment, prevention or alleviation of a disease or a disorder or a condition of a living animal body, including a human, which disorder, disease or condition is responsive to positive modulation of AMPA receptor mediated synaptic responses, which method comprises the step of administering to such a living animal body in need thereof, a therapeutically effective amount of the quinoxaline derivative of the invention.

Other objects of the invention will be apparent to the person skilled in the art from the following detailed description and examples.

### DETAILED DISCLOSURE OF THE INVENTION

The quinoxaline derivatives of the invention may be characterised by Formula I

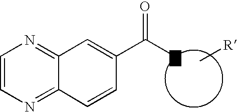

any of its enantiomers or any mixture of its enantiomers, or a pharmaceutically-acceptable addition salt thereof, or an N-oxide thereof, wherein the circular line designates an N-containing heterocyclic 5-8 membered ring (i.e. a pyrrolidin-1-yl, a piperidin-1-yl, an azepan-1-yl or an azocan-1-yl group); and R′ represents 2- or 3-alkyl, cycloalkyl, hydroxy, alkoxy, alkoxy-alkyl, hydroxy-alkyl or carbamoyl.

In a preferred embodiment of the invention the circle line designates a pyrrolidin-1-yl, azepan-1-yl or azocan-1-yl group; and R′ represents hydrogen, alkyl, cycloalkyl, hydroxy, alkoxy, alkoxy-alkyl, hydroxy-alkyl or carbamoyl; or the circle line designates a piperidin-1-yl group; and R′ represents 2- or 3-alkyl, cycloalkyl, hydroxy, alkoxy, alkoxy-alkyl, hydroxy-alkyl or carbamoyl.

In a more preferred embodiment R′ represents alkyl or cycloalkyl.

In an even more preferred embodiment R′ represents 2- or 3-alkyl, or 2- or 3-cycloalkyl.

In a still more preferred embodiment R′ represents methyl, ethyl or cyclopropyl.

In a second preferred embodiment the circle line designates a pyrrolidin-1-yl group; and R′ represents hydrogen, alkyl, cycloalkyl, hydroxy, alkoxy, alkoxy-alkyl, hydroxy-alkyl or carbamoyl.

In a more preferred embodiment the circle line designates a pyrrolidin-1-yl group; and R′ represents hydrogen, methyl, ethyl, cyclopropyl, hydroxy, methoxy, ethoxy, methoxy-methyl, ethoxy-methyl, methoxy-ethyl, ethoxy-ethyl, hydroxy-methyl or hydroxy-ethyl.

In an even more preferred embodiment the circle line designates a pyrrolidin-1-yl group; and R′ represents hydrogen, alkyl or cycloalkyl.

In a still more preferred embodiment the circle line designates a pyrrolidin-1-yl group; and R′ represents hydrogen, methyl, ethyl or cyclopropyl.

In a yet more preferred embodiment the circle line designates a pyrrolidin-1-yl group; and R′ represents alkyl or cycloalkyl.

In a further preferred embodiment the circle line designates a pyrrolidin-1-yl group; and R′ represents methyl, ethyl or cyclopropyl.

In a most preferred embodiment the quinoxaline derivative of the invention is

Pyrrolid in-1-yl-quinoxalin-6-yl-methanone;

(±)-(2-Methyl-pyrrolidin-1-yl)-quinoxalin-6-yl-methanone; or

(±)-(2-Ethyl-pyrrolidin-1-yl)-quinoxalin-6-yl-methanone;

or a pharmaceutically-acceptable addition salt thereof.

In a third preferred embodiment of the invention the circle line designates a piperidin-1-yl group; and R′ represents 2- or 3-alkyl, cycloalkyl, hydroxy, alkoxy, alkoxy-alkyl, hydroxy-alkyl or carbamoyl.

In a more preferred embodiment the circle line designates a piperidin-1-yl group; and R′ represents 2-methyl, 2-ethyl, 3-methyl, 3-ethyl, hydroxy, methoxy, ethoxy, methoxy-methyl, ethoxy-methyl, hydroxy-methyl or carbamoyl.

In an even more preferred embodiment the circle line designates a piperidin-1-yl group; and R′ represents 2-methyl, 2-ethyl, 2-cyclopropyl, 3-methyl, 3-ethyl, 3-cyclopropyl, hydroxy, methoxy, ethoxy, methoxy-methyl, ethoxy-methyl, methoxy-ethyl, ethoxy-ethyl, hydroxy-methyl or hydroxy-ethyl.

In a still more preferred embodiment the circle line designates a piperidin-1-yl group; and R′ represents 2-methyl, 2-ethyl, 3-methyl, 3-ethyl, hydroxy, methoxy, ethoxy, methoxy-methyl, ethoxy-methyl or hydroxy-methyl or carbamoyl.

In a further more preferred embodiment the circle line designates a piperidin-1-yl group; and R′ represents 2-alkyl, 3-alkyl, 2-cycloalkyl or 3-cycloalkyl.

In a still further preferred more embodiment the circle line designates a piperidin-1-yl group; and R′ represents 2-methyl, 2-ethyl, 2-cyclopropyl, 3-methyl, 3-ethyl or 3-cyclopropyl.

In a most preferred embodiment the quinoxaline derivative of the invention is

(±)-(2-Methyl-piperidin-1-yl)-quinoxalin-6-yl-methanone;

(±)-(2-Ethyl-piperidin-1-yl)-quinoxalin-6-yl-methanone;

(±)-(3-Methyl-piperidin-1-yl)-quinoxalin-6-yl-methanone;

(±)-(3-Ethyl-piperidin-1-yl)-quinoxalin-6-yl-methanone;

(±)-(3-Hydroxy-piperidin-1-yl)-quinoxalin-6-yl-methanone;

(±)-(3-Hydroxy-methyl-piperidin-1-yl)-quinoxalin-6-yl-methanone;

(±)-(3-Methoxy-piperidin-1-yl )-quinoxalin-6-yl-methanone;

(±)-(3-Ethoxy-piperidin-1-yl)-quinoxalin-6-yl-methanone;

(±)-(3-Methoxy-methyl-piperidin-1-yl )-quinoxalin-6-yl-methanone;

(±)-(3-Ethoxy-methyl-piperidin-1-yl)-quinoxalin-6-yl-methanone; or

(±)-(3-Carbamoyl-piperidin-1-yl)-quinoxalin-6-yl-methanone;

or a pharmaceutically-acceptable addition salt thereof.

In a particularly preferred embodiment the quinoxaline derivative of the invention is

(±)-(3-Methyl-piperidin-1-yl)-quinoxalin-6-yl-methanone;

or a pharmaceutically-acceptable addition salt thereof.

In a fourth preferred embodiment of the invention the circle line designates an azepan-1-yl or azocan-1-yl group; and R′ represents hydrogen, alkyl, cycloalkyl, hydroxy, alkoxy, alkoxy-alkyl, hydroxy-alkyl or carbamoyl.

In a more preferred embodiment the circle line designates an azepan-1-yl group; and R′ represents hydrogen, alkyl or cycloalkyl.

In another preferred embodiment the circle line designates an azocan-1-yl group; and R′ represents hydrogen, alkyl or cycloalkyl.

In a most preferred embodiment the quinoxaline derivative of the invention is

Azepan-1-yl-quinoxalin-6-yl-methanone; or

Azocan-1-yl-quinoxalin-6-yl-methanone;

or a pharmaceutically-acceptable addition salt thereof.

Any combination of two or more of the embodiments described herein is considered within the scope of the present invention.

### Definition of Substituents

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention a cycloalkyl group designates a cyclic alkyl group, preferably containing of from three to seven carbon atoms (C₃₋₇-cycloalkyl), including cyclopropyl, cyclobutyl, cyclopentyl, cyclohexyl and cycloheptyl.

In the context of this invention an alkoxy group designates an “alkyl-O—” group, wherein alkyl is as defined above. Examples of preferred alkoxy groups of the invention include methoxy and ethoxy.

In the context of this invention an alkoxy-alkyl group designates an “alkyl-O— alkyl-” group, wherein alkyl is as defined above. Examples of preferred alkoxy-alkyl groups of the invention include methoxy-methyl, methoxy-ethyl, ethoxy-methyl, and ethoxy-ethyl.

In the context of this invention a hydroxy-alkyl group designates an alkyl group as defined above, which hydroxy-alkyl group is substituted with one or more hydroxy groups. Examples of preferred hydroxy-alkyl groups of the invention include 2-hydroxy-ethyl, 3-hydroxy-propyl, 4-hydroxy-butyl, 5-hydroxy-pentyl and 6-hydroxy-hexyl.

### Pharmaceutically Acceptable Salts

The quinoxaline derivative of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrohloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a chemical compound of the invention include, without limitation, the sodium, the potassium, the calcium, the magnesium, the zinc, the aluminium, the lithium, the choline, the lysine, and the ammonium salt, and the like, of a chemical compound of the invention containing an anionic group. Such cationic salts may be formed by procedures well known and described in the art.

Other examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Examples of pharmaceutically acceptable cationic salts of a chemical compound of the invention include, without limitation, the sodium, the potassium, the calcium, the magnesium, the zinc, the aluminium, the lithium, the choline, the lysine, and the ammonium salt, and the like, of a chemical compound of the invention containing an anionic group. Such cationic salts may be formed by procedures well known and described in the art.

### Steric Isomers

The quinoxaline derivatives of the present invention may exist in (+) and (−) forms as well as in racemic forms (±). The racemates of these isomers and the individual isomers themselves are within the scope of the present invention.

Racemic forms can be resolved into the optical antipodes by known methods and techniques. One way of separating the diastereomeric salts is by use of an optically active acid, and liberating the optically active amine compound by treatment with a base. Another method for resolving racemates into the optical antipodes is based upon chromatography on an optical active matrix. Racemic compounds of the present invention can thus be resolved into their optical antipodes, e.g., by fractional crystallisation of d- or I- (tartrates, mandelates, or camphorsulphonate) salts for example.

The quinoxaline derivatives of the present invention may also be resolved by the formation of diastereomeric amides by reaction of the chemical compounds of the present invention with an optically active activated carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials.

### Methods of Preparation

The quinoxaline derivative of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

The quinoxaline derivatives of the invention have shown potential for positive modulation of AMPA receptor mediated synaptic responses. More particularly it has been found that the quinoxaline derivatives of the invention show a less profound desensitization of glutamate receptors and have the potential for prolonging synaptic responses mediated through activation of AMPA receptors.

AMPA receptors have been associated with diseases and conditions as diverse as cognitive deficits, intellectual impairment disorders, memory deficit, learning deficit, memory loss, psychotic disorders, sexual dysfunction, amyotrophic lateral sclerosis (ALS), multiple schlerosis (MS), schizophrenia, depression, autism, Alzheimer's disease, attention deficit hyperactivity disorder (ADHD), senile dementia, disorders or disease resulting from trauma, stroke, epilepsy, Alzheimer's disease, neurotoxic agents, aging, neurodegenerative disorder, alcohol intoxication, substance abuse, cardiac bypass surgery, and cerebral ischaemia.

In a preferred embodiment the disease or disorder or condition is selected from the group consisting of cognitive disorders, memory and learning disorders, schizophrenia, cognitive disorders associated with schizophrenia, depression, attention deficit hyperactivity disorder, neurodegenerative conditions, in particular neurodegenerative conditions associated with ageing, Alzheimers disease, mild cognitive impairment, dementias associated with multiple schlerosis or Parkinson's disease, and anxiety disorders.

In another preferred embodiment the disease or disorder or condition is a cognitive disorder, memory or learning disorder, schizophrenia and cognitive disorders associated with schizophrenia.

### Pharmaceutical Compositions

In another aspect the invention provides novel pharmaceutical compositions comprising a therapeutically effective amount of the quinoxaline derivative.

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the quinoxaline derivative, or a pharmaceutically acceptable salt or derivative thereof, together with one or more pharmaceutically acceptable carriers, and, optionally, other therapeutic and/or prophylactic ingredients, known and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be prepared by any person skilled in the art, by use of standard methods and conventional techniques, appropriate to the desired formulation. When desired, compositions adapted to give sustained release of the active ingredient may be employed.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

In another aspect the invention provides a method for the treatment, prevention or alleviation of a disease or a disorder or a condition of a living animal body, including a human, which disease, disorder or condition is responsive to positive modulation of AMPA receptor mediated synaptic responses.

In the context of this invention the term “treatment” covers treatment, prevention, prophylaxis or alleviation, and the term “disease” covers illnesses, diseases, disorders and conditions related to the disease in question.

The preferred indications contemplated according to the invention are those stated above.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

### BRIEF DESCRIPTION OF THE DRAWINGS

The present invention is further illustrated by reference to the accompanying drawing, in which:

FIG. 1 shows the activity of 10 mM glutamate in the absence (control) and presence of a compound of the prior art (Reference compound; Piperidin-1-yl-quinoxalin-6-yl-methanone; WO 9402475), determined as “normalized current” (in the range 0.0 and 1.0), for a period of up to 0.05 seconds (0.00-0.05 s), at a concentration of 1 mM of test compound, on a Chinese Hamster Ovary (CHO) cell capable of over-expressing the human glutamate receptor flip variant (hGluR1(i)); and

FIG. 2 shows the activity of 10 mM glutamate in the absence (control) and presence of a compound representative of the invention (i.e. (3-Methyl-piperidin-1-yl)-quinoxalin-6-yl-methanone; Compound A4), determined as “normalized current” (in the range 0.0 and 1.0), for a period of up to 0.05 seconds (0.00-0.05 s), at a concentration of 1 mM of test compound, on a Chinese Hamster Ovary (CHO) cell capable of over-expressing the human glutamate receptor flip variant (hGluR1(i)).

### EXAMPLES

The invention is further illustrated with reference to the following examples, which are not intended to be in any way limiting to the scope of the invention as claimed.

### Example 1

### Preparatory Example

Quinoline-6-carboxylic Acid (Intermediate Compound)

To a mixture of 3,4-diaminobenzoic acid (30.0 g; 197 mmol) and ethanol (300 ml; 99%), was added: a mixture of 40% aqueous glyoxal (33 ml; 227 mmol) in ethanol (75 ml, 99%) was added at room temperature. The mixture was allowed to stir overnight at room temperature. The product was isolated as a grey powder by filtration. Yield 24.4 g (71%).

### Method A

Pyrrolidin-1-yl-guinoxalin-6-yl-methanone Hydrochloric Acid Salt (Compound A1)

A mixture of quinoline-6-carboxylic acid (3.48 g; 20 mmol) and thionylchloride (10 ml; 137 mmol) was stirred at 60° C. for 4 hours. The mixture was allowed to cool to room temperature. The solid quinoline-6-carboxylic acid chloride was washed with diethyl ether.

Pyrrolidine (3.13 g; 44 mmol) was added to a mixture of quinoline-6-carboxylic acid chloride (3.8 g; 20 mmol) and THF (50 ml). The mixture was allowed to stir at room temperature overnight. The brown oil was purified by silica gel chromatography and ethylacetate+20% methanol as eluent. Yield 0.97 g (22%). The oil was converted to the corresponding hydrochloric acid salt by stirring overnight in a mixture of HCl in ethanol and diethylether. Mp. 115.7° C.

(±)-(2-Ethyl-piperidin-1-yl)-guinoxalin-6-yl-methanone Hydrochloric Acid Salt (Compound A2)

Was prepared according to Method A from (±)-2-ethylpiperidine. Mp. 108.9-110.2° C.

(±)-(2-Methyl-piperidin-1-yl)-guinoxalin-6-yl-methanone Hydrochloric Acid Salt (Compound A3)

Was prepared according to Method A from (±)-2-methylpiperidine. Mp. 130-135° C.

(±)-(3-Methyl-piperidin-1-yl)-guinoxalin-6-yl-methanone Hydrochloric Acid Salt (Compound A4)

Was prepared according to Method A from (±)-3-methylpiperidine. Mp. 95-100° C.

(±)-(2-Methyl-piperidin-1-yl)-guinoxalin-6-yl-methanone Hydrochloric Acid Salt (Compound A5)

Was prepared according to Method A from (±)-2-methylpyrrolidine. Mp. 130-132° C.

(±)-(3-Methyl-piperidin-1-yl)-guinoxalin-6-yl-methanone Free Base (Compound A6)

Was prepared according to Method A from (±)-3-piperidine methanol. LC-ESI-HRMS of [M+H]+ shows 272.1393 Da. Calc. 272.139902 Da, dev. 31 2.2 ppm.

Azepan-1-yl-quinoxalin-6-yl-methanone Hydrochloric Acid Salt (Compound A7)

Was prepared according to Method A from hexamethyleneimine. LC-ESI-HRMS of [M+H]+ shows 256.1442 Da. Calc. 256.144987 Da, dev. −3.1 ppm.

Azocan-1-yl-quinoxalin-6-yl-methanone Free Base (Compound A8)

Was prepared according to Method A from heptaethyleneimine. LC-ESI-HRMS of [M+H]+ shows 270.1593 Da. Calc. 270.160637 Da, dev. −4.9 ppm.

(±)-(3-Hydroxy-piperidin-1-yl)-guinoxalin-6-yl-methanone Free Base (Compound A9)

Was prepared according to Method A from (±)-3-hydroxypiperidine. LC-ESI-HRMS of [M+H]+ shows 258.1237 Da. Calc. 258.124252 Da, dev. −2.1 ppm.

(±)-1-(Quinoxaline-6-carbonyl)-piperidine-3-carboxylic Acid Amide Free Base (Compound A10)

Was prepared according to Method A from (±)-(3-piperidinecarboxamide.

### Method B

(±)-(3-Ethoxymethyl-piperidin-1-yl)-guinoxalin-6-yl-methanone Free Base (Compound B1)

A mixture of (±)-(3-hydroxymethyl-piperidin-1-yl)-quinoxalin-6-yl-methanone (1.5 g, 4.09 mmol), bromoethane (0.44 g, 4.09 mmol), potassium tert-butoxide (1.38 g, 12.3 mmol) and dioxane (20 ml) was stirred at 70° C. for 3 h. The mixture was cooled to room temperature. Water (20 ml) was added and the mixture was extracted with diethylether (2×30 ml). The mixture was dried and evaporated. Yield 0.25 g (21%). LC-ESI-HRMS of [M+H]+shows 300.1717 Da. Calc. 300.171202 Da, dev. 1.7 ppm.

(±)-(3-Methoxymethyl-piperidin-1-yl)-guinoxalin-6-yl-methanone Free Base (Compound B2)

Was prepared according to Method B from (±)-(3-hydroxymethyl-piperidin-1-yl)-quinoxalin-6-yl-methanone and iodomethane at room temperature. LC-ESI-HRMS of [M+H]+ shows 286.1544 Da. Calc. 286.155552 Da, dev. −4 ppm.

(±)-(3-Ethoxy-piperidin-1-yl)quinoxalin-6-yl-methanone Free Base (Compound B3)

Was prepared according to Method B from (±)-(3-hydroxy-piperidin-1-yl)-quinoxalin-6-yl-methanone and bromoethane.

(±)-(3-Methoxy-piperidin-1-yl)-guinoxalin-6-yl-methanone Free Base (Compound B4)

Was prepared according to Method B from (±)-(3-hydroxy-piperidin-1-yl)-quinoxalin-6-yl-methanone and iodomethane.

### Example 2

### Functional Activity

In this example a compound, i.e. (3-Methyl-piperidin-1-yl)-quinoxalin-6-yl-methanone (Compound A4), representative of the quinoxaline derivatives of the invention, is compared to a quinoxaline derivative of the prior art (Reference compound; Piperidin-1-yl-quinoxalin-6-yl-methanone, described in WO 9402475) with respect to its activity on a glutamate receptor.

AMPA receptors are approximately 900 amino acids in length and occur in two forms distinguished by the presence or absence of an alternatively spliced exonic sequence of 38 residues preceding the last transmembrane domain. This alternative splicing of GluR1-GluR4 results in the socalled “flip” or “flop” variants. The “flip” forms give rise to a larger sustained current (slower to desensitize) than do the “flop” forms.

In this experiment we investigated the influence of the test compounds on the membrane currents when determined electrophysiologically on Chinese Hamster Ovary (CHO) cells capable of expressing the human glutamate receptor GluR1-flip, obtained essentially as described by Fletcher E J & Lodge D, Pharmacol. Ther. 1996 70 (1) 65-89, the current through the channels being recorded using patch clamp technique in whole cell voltage clamp mode.

Current responses were induced by applying L-glutamate at a saturating concentration of 10 mM, and the modulatory effect of the test compound was evaluated by co-applying the test compound and L-glutamate upon a 30 seconds pre-incubation period, and comparing with the test compound alone.

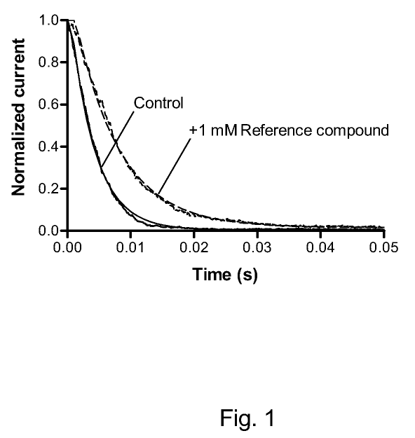

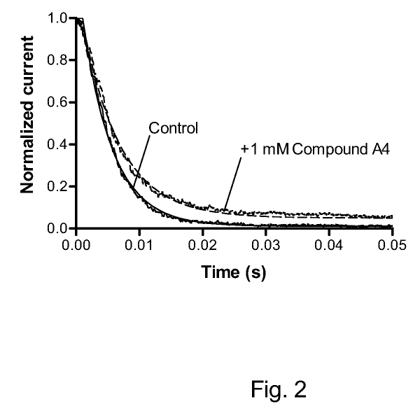

The results of these determinations are presented in FIGS. 1-2.

In FIG. 1 the degree of desensitization shifts from 98.7+/−1.1% to 98.0+/−0.8% in the presence of 1 mM compound of the Reference compound.

In FIG. 2 the degree of desensitization shifts from 98.9+/−0.0% to 95.2+/−0.2% in the presence of 1 mM compound of the invention (Compound A4).

From this comparison it shows that the compound of the invention reduces the rate of desensitization and, comparison with the quinoxaline derivative of the prior art, the desensitization becomes less profound.

1-18. (canceled)

19. A quinoxaline derivative of Formula I 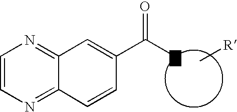
any of its stereoenantiomers or any mixture of its stereoenantiomers, or a pharmaceutically-acceptable addition salt thereof, or an N-oxide thereof, wherein
the circle line designates a pyrrolidin-1-yl, azepan-1-yl or azocan-1-yl group; and
R′ represents hydrogen, alkyl, cycloalkyl, hydroxy, alkoxy, alkoxy-alkyl, hydroxy-alkyl or carbamoyl; or
the circle line designates a piperidin-1-yl group; and
R′ represents 2- or 3-alkyl, cycloalkyl, hydroxy, alkoxy, alkoxy-alkyl, hydroxy-alkyl or carbamoyl.

20. The quinoxaline derivative of claim 19, or a pharmaceutically-acceptable addition salt thereof, wherein
R′ represents methyl, ethyl or cyclopropyl.

21. The quinoxaline derivative of claim 19, or a pharmaceutically-acceptable addition salt thereof, wherein
the circle line designates a pyrrolidin-1-yl group; and
R′ represents hydrogen, alkyl, cycloalkyl, hydroxy, alkoxy, alkoxy-alkyl, hydroxy-alkyl or carbamoyl.

22. The quinoxaline derivative of claim 21, or a pharmaceutically-acceptable addition salt thereof, wherein
the circle line designates a pyrrolidin-1-yl group; and
R′ represents hydrogen, alkyl or cycloalkyl.

23. The quinoxaline derivative of claim 22, or a pharmaceutically-acceptable addition salt thereof, wherein
the circle line designates a pyrrolidin-1-yl group; and
R′ represents alkyl or cycloalkyl.

24. The quinoxaline derivative of claim 21, which is
Pyrrolidin-1-yl-quinoxalin-6-yl-methanone;
(±)-(2-Methyl-pyrrolidin-1-yl)-quinoxalin-6-yl-methanone; or
(±)-(2-Ethyl-pyrrolidin- 1-yl)-quinoxalin-6-yl-methanone;
or a pharmaceutically-acceptable addition salt thereof.

25. The quinoxaline derivative of claim 19, or a pharmaceutically-acceptable addition salt thereof, wherein
the circle line designates a piperidin-1-yl group; and
R′ represents 2- or 3-alkyl, cycloalkyl, hydroxy, alkoxy, alkoxy-alkyl, hydroxy-alkyl or carbamoyl.

26. The quinoxaline derivative of claim 25, or a pharmaceutically-acceptable addition salt thereof, wherein
the circle line designates a piperidin-1-yl group; and
R′ represents 2-methyl, 2-ethyl, 3-methyl, 3-ethyl, hydroxy, methoxy, ethoxy, methoxy-methyl, ethoxy-methyl, hydroxy-methyl or carbamoyl.

27. The quinoxaline derivative of claim 25, which is
(±)-(2-Methyl-piperidin-1-yl)-quinoxalin-6-yl-methanone;
(±)-(2-Ethyl-piperidin-1-yl)-quinoxalin-6-yl-methanone;
(±)-(3-Methyl-piperidin-1-yl)-quinoxalin-6-yl-methanone;
(±)-(3-Ethyl-piperidin-1-yl)-quinoxalin-6-yl-methanone;
(±)-(3-Hydroxy-piperidin-1-yl)-quinoxalin-6-yl-methanone;
(±)-(3-Hydroxy-methyl-piperidin-1-yl)-quinoxalin-6-yl-methanone;
(±)-(3-Methoxy-piperidin-1-yl)-quinoxalin-6-yl-methanone;
(±)-(3-Ethoxy-piperidin-1-yl)-quinoxalin-6-yl-methanone;
(±)-(3-Methoxy-methyl-piperidin-1-yl)-quinoxalin-6-yl-methanone;
(±)-(3-Ethoxy-methyl-piperidin-1-yl)-quinoxalin-6-yl-methanone; or
(±)-(3-Carbamoyl-piperidin-1-yl)-quinoxalin-6-yl-methanone;
or a pharmaceutically-acceptable addition salt thereof.

28. The quinoxaline derivative of claim 19, or a pharmaceutically-acceptable addition salt thereof, wherein
the circle line designates an azepan-1-yl or azocan-1-yl group; and
R′ represents hydrogen, alkyl, cycloalkyl, hydroxy, alkoxy, alkoxy-alkyl, hydroxy-alkyl or carbamoyl.

29. The quinoxaline derivative of claim 28, or a pharmaceutically-acceptable addition salt thereof, wherein
the circle line designates an azepan-1-yl or azocan-1-yl group; and
R′ represents hydrogen, alkyl or cycloalkyl.

30. The quinoxaline derivative of claim 28, which is
Azepan-1-yl-quinoxalin-6-yl-methanone; or
Azocan-1-yl-quinoxalin-6-yl-methanone;
or a pharmaceutically-acceptable addition salt thereof.

31. A pharmaceutical composition comprising a therapeutically effective amount of the quinoxaline derivative of claim 19, or a pharmaceutically-acceptable addition salt thereof.

32. A method of treatment, prevention or alleviation of a disease or a disorder or a condition of a living animal body, including a human, which disorder, disease or condition is responsive to positive modulation of AMPA receptor mediated synaptic responses, which method comprises the step of administering to such a living animal body in need thereof, a therapeutically effective amount of the quinoxaline derivative of claim 19.

33. The method according to claim 32, wherein the disease, disorder or condition is selected from the group consisting of cognitive disorders, memory and learning disorders, psychotic disorders, sexual dysfunction, amyotrophic lateral sclerosis (ALS), multiple schlerosis (MS), intellectual impairment disorders, schizophrenia, depression, autism, Alzheimer's disease, learning deficit, attention deficit hyperactivity disorder (ADHD), memory loss, senile dementia, disorders or disease resulting from trauma, stroke, epilepsy, Alzheimer's disease, neurotoxic agents, aging, neurodegenerative disorder, alcohol intoxication, substance abuse, cardiac bypass surgery and cerebral ischaemia.

34. The method according to claim 32, wherein the disease, disorder or condition is selected from the group consisting of cognitive disorders, memory and learning disorders, schizophrenia, cognitive disorders associated with schizophrenia, depression, attention deficit hyperactivity disorder, neurodegenerative conditions, in particular neurodegenerative conditions associated with ageing, Alzheimers disease, mild cognitive impairment, dementias associated with multiple schlerosis or Parkinson's disease, and anxiety disorders.
